(19) United States
(12) Patent Application Publication
(10) Pub. No.: US 2022/0186296 A1
(43) Pub. Date: Jun. 16, 2022

(54) MULTIPLEXED SIGNAL AMPLIFICATION METHODS USING ENZYMATIC BASED CHEMICAL DEPOSITION

(71) Applicant: The Board of Trustees of the Leland Stanford Junior University, Stanford, CA (US)

(72) Inventors: Garry P. NOLAN, Redwood City, CA (US); Xavier ROVIRA CLAVE, Menlo Park, CA (US); Sizun Jiang, Stanford, CA (US); Yunhao Bai, Stanford, CA (US); Tung-Hung Su, Stanford, CA (US); Shih-yu Chen, Redwood City, CA (US)

(21) Appl. No.: 17/433,542

(22) PCT Filed: Feb. 25, 2020
(86) PCT No.: PCT/US20/19740

§ 371 Date: Aug. 24, 2021

Related U.S. Application Data
(60) Provisional application No. 62/811,993, filed on Feb. 28, 2019.

(51) Int. Cl. C12Q 1/6818; C12N 9/96; C12Q 1/6841

(52) U.S. Cl. CPC C12Q 1/6818; C12Q 1/6841; C12N 9/96

(57) ABSTRACT

Provided herein, among other things, is a method for analyzing a sample. In some embodiments, the method makes use of a plurality of binding agents that are each linked to a different oligonucleotide, as well as a corresponding plurality of peroxidase-linked oligonucleotides, wherein each of the peroxidase-linked oligonucleotides specifically hybridizes with only one of the binding agent-linked oligonucleotides. In some embodiments the method may comprise labeling the sample with the plurality of binding agents en masse, and then staining the sample by hybridizing a single peroxidase-linked oligonucleotide with the sample to produce complexes that comprise the peroxidase and then treating the sample with at least one tyramide-label conjugate. The peroxidase in the complexes activates the conjugate and causes covalent binding of the label to the sample near the complexes. Reagents and kits for performing the method are also provided.

### CROSS-REFERENCING

This application claims the benefit of U.S. provisional application Ser. No. 62/811,993, filed on Feb. 28, 2019, which application is incorporated by reference in its entirety.

### BACKGROUND

In tyramide signal amplification (TSA), also known as catalyzed reporter deposition (CARD), a peroxidase (e.g., HRP) converts a tyramide-label conjugate (i.e., tyramide that has been labeled with, e.g., a fluorophore or hapten such as biotin) into a highly reactive product that can covalently bind to tyrosine residues on proteins at or near the peroxidase. Each peroxidase molecule causes several molecules of the tyramide-conjugate to be deposited locally to the enzyme molecule, thereby resulting in dense labeling around the site of the enzyme. This dense labeling makes tyramide signal amplification more sensitive than other commonly used methods. Tyramide signal amplification is described in Bobrow et al. (J. Immunol. Methods 1989 125: 279-285) and Bobrow et al. (J. Immunol. Methods 1991 137 103-112), among other publications.

However, labeling methods that rely on tyramide signal amplification often suffer from similar problems as conventional immunohistochemistry methods in that the number of different epitopes that can be analyzed is limited by the spectral properties of the labels used. Specifically, while tyramide signal amplification has been successfully used to label three epitopes simultaneously using three distinguishable fluorescent labels (see, e.g., Mitchell et al Mod. Pathol. 2014 27:1255-1266 and Toth J. Histochem. Cytochem. 2007 55 545-554), four does not appear to have been achieved. Based on current technology, it would be challenging to analyze more than three epitopes using tyramide signal amplification labeling system, much less more than 5 or 10 epitopes. This constraint is problematic because it limits the use of tyramide signal amplification in clinical diagnostics, in which field it is very desirable to analyze a much larger number of epitopes.

### SUMMARY

Provided herein, among other things, is a method for analyzing a sample. In some embodiments, the method makes use of a plurality of binding agents that are each linked to a different oligonucleotide, as well as a corresponding plurality of peroxidase-linked oligonucleotides, wherein each of the peroxidase-linked oligonucleotides specifically hybridizes with only one of the binding agent-linked oligonucleotides. In some embodiments, the method may comprise labeling the sample with the plurality of binding agents en masse, and then staining the sample by hybridizing a single peroxidase-linked oligonucleotide with the sample to produce complexes that comprise the peroxidase and then treating the sample with at least one tyramide-label conjugate. The peroxidase in the complexes activates the conjugate and cause covalent binding of the label to the sample near the complexes.

The label in the conjugate may be, e.g., a fluorophore, mass tag or heavy metal and the sample may be analyzed using any of a variety of different methods, e.g., mass-cytometry, multiplexed ion beam imaging (MIBI), fluorescence microscopy or electron microscopy.

### BRIEF DESCRIPTION OF THE FIGURES

The skilled artisan will understand that the drawings described below are for illustration purposes only. The drawings are not intended to limit the scope of the present teachings in any way.

FIG. 1 is a flow chart illustrating how some embodiments of the present method can be implemented.

FIG. 2 illustrates how tyramide signal amplification can be done using a tyramide-mass tag conjugate.

FIG. 3 illustrates how spatial barcoding (i.e., labeling the same site in a sample with a combination of distinguishable labels, rather than a single label) can be achieved using tyramide-mass tag conjugates.

FIG. 4 illustrates how a tyramide-chelater conjugate can be produced.

FIG. 5 shows the results of a mass spectrometry analysis of the product of the reaction shown in FIG. 4. This data indicates that the reaction produces a relatively high yield of DOTA-tyramide.

FIG. 6 illustrates how a tyramide-chelater conjugate can be loaded with a lanthanide mass tag (e.g., ¹⁵¹Eu or ¹⁶¹Dy) to produce a tyramide-mass tag conjugate.

FIG. 7 shows the results of a mass spectrometry analysis of the reaction shown in FIG. 6. This data indicates that the reaction produces a relatively high yield of the product.

FIG. 8 schematically illustrates how the present method can be implemented by in situ hybridization and fluorescently-labeled or mass tag-labeled oligonucleotides. In this design, oligo-HRP conjugates are used in combination with a conjugate containing an Alexa Fluor® 488 fluorescent tag or a 151-Eu mass tag. A primary DNA probe (grey) is first hybridized with cells to bind to the DNA target of interest (alpha-satellite DNA repeat sequences in this case). A secondary oligo is used to detect the primary probe. In this case, either an Alexa Fluor® 647 probe is used or an HRP secondary probe is used. If the HRP-secondary probe is used then the sample can be stained by treatment with a tyramide-fluorophore conjugate or tyramide-lanthanide conjugate.

FIG. 9 shows experimental results obtained by fluorescence microscopy using an Alexa Fluor® 647 secondary probe (left) and Alexa Fluor® 488 deposited by an oligo-HRP conjugate. Staining is for alpha-satellite DNA repeats. Cells used are FFPE embedded cell pellets from HeLa cells.

FIG. 10 shows experimental results obtained by MIBI, where 151-Eu is deposited by an oligo-HRP conjugate as described above. DNA staining is for alpha-satellite DNA repeats. In addition, Histone H3 staining was performed to demarcate the nucleus (shown in blue).

FIG. 11 shows experimental results obtained by fluorescence microscopy (left) and MIBI (right), where the probes detect single-SIV viral integration events. The cells used in these experiments are FFPE embedded 3D8 T cell pellets.

FIG. 12 shows experimental results obtained by mass spectrometry (CyTOF), where the nucleolin in K562 cells is stained using a tyramide-lanthanide conjugate. K562 cells were stained with anti-nucleolin primary antibody, and then a secondary HRP antibody. TSA-lanthanide was used for chemical deposition in the presence (bottom) or absence (top) of primary antibody staining.

FIG. 13 shows experimental results obtained by fluorescence microscopy showing the specificity of labeling and that oligo-HRP deposition can be done sequentially. Cells used are HeLa cells. These results show that barcoded oligo-HRP enables sequential deposition of fluorophore or lanthanide tags.

FIG. 14 illustrates how a heavy metal (silver, Ag) can be deposited using HRP.

FIG. 15 illustrates how heavy metals deposited via HRP can be analyzed by electron microscopy (e.g., scanning electron microscopy (SEM)). In this experiment, HeLa cells were labeled with anti-lamin antibodies, then with a secondary-HRP antibody Images shown were performed on a scanning electron microscope. The cells used are HeLa cells.

FIG. 16 illustrates two examples of tyramide-flourophore conjugates that are cleavable by TCEP.

FIG. 17 illustrates the chemistry that can be used to link an oligonucleotide and a peroxidase.

### DEFINITIONS

Unless defined otherwise herein, all technical and scientific terms used in this specification have the same meaning as commonly understood by one of ordinary skill in the art to which this invention belongs. Although any methods and materials similar or equivalent to those described herein can be used in the practice or testing of the present invention, the preferred methods and materials are described.

All patents and publications, including all sequences disclosed within such patents and publications, referred to herein are expressly incorporated by reference.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention. Accordingly, the terms defined immediately below are more fully defined by reference to the specification as a whole.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of ordinary skill in the art with the general meaning of many of the terms used herein. Still, certain terms are defined below for the sake of clarity and ease of reference.

As used herein, the term “biological feature of interest” refers to any part of a cell that can be indicated by binding to a binding agent. Exemplary biological features of interest include cell walls, nuclei, cytoplasm, membrane, keratin, muscle fibers, collagen, bone, proteins, nucleic acid (e.g., mRNA or genomic DNA, etc.), etc. A binding agent may bind to a corresponding site, e.g., a protein epitope, in the sample.

As used herein, the term “multiplexing” refers to the simultaneous detection and/or measurement of multiple biological features of interest, e.g., protein epitopes, in a sample.

As used herein, the terms “antibody” and “immunoglobulin” are used interchangeably herein and are well understood by those in the field. Those terms refer to a protein consisting of one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions.

The terms “antibodies” and “immunoglobulin” include antibodies or immunoglobulins of any isotype and fragments of antibodies which retain specific binding to antigen, including, but not limited to, Fab, Fv, scFv, and Fd fragments, chimeric antibodies, humanized antibodies, minibodies, single-chain antibodies, and fusion proteins comprising an antigen-binding portion of an antibody and a non-antibody protein. Also encompassed by the term are Fab′, Fv, F(ab′)₂, and/or other antibody fragments that retain specific binding to antigen, and monoclonal antibodies. Antibodies may exist in a variety of other forms including, for example, Fv, Fab, and (Fab′)₂, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988)), which are incorporated herein by reference. (See, generally, Hood et al., “Immunology”, Benjamin, N.Y., 2nd ed. (1984), and Hunkapiller and Hood, Nature, 323, 15-16 (1986)).

The term “specific binding” refers to the ability of a binding agent to preferentially bind to a particular analyte that is present in a homogeneous mixture of different analytes. In certain embodiments, a specific binding interaction will discriminate between desirable and undesirable analytes in a sample. In some embodiments, more than about 10- to 100-fold or more (e.g., more than about 1000- or 10,000-fold).

In certain embodiments, the affinity between a binding agent and analyte when they are specifically bound in a binding agent/analyte complex is characterized by a K_{D} (dissociation constant) of less than 10⁻⁶M, less than 10⁻⁷ M, less than 10⁻⁸ M, less than 10⁻⁹ M, less than 10⁻⁹ M, less than 10⁻¹¹ M, or less than about 10⁻¹² M or less.

A “plurality” contains at least 2 members. In certain cases, a plurality may have at least 2, at least 5, at least 10, at least 100, at least 1000, at least 10,000, at least 100,000, at least 10⁶, at least 10⁷, at least 10⁸ or at least 10⁹ or more members. In certain cases, a plurality may have 2 to 100 or 5 to 100 members.

As used herein, the term “labeling” refers to a step that results in binding of a binding agent to specific sites in a sample (e.g., sites containing an epitope for the binding agent (e.g., an antibody) being used, for example) such that the presence and/or abundance of the sites can be determined by evaluating the presence and/or abundance of the binding agent. The term “labeling” refers to a method for producing a labeled sample in which any necessary steps are performed in any convenient order, as long as the required labeled sample is produced. For example, in some embodiments and as will be exemplified below, a sample can be labeled using a plurality of binding agents that are each linked to an oligonucleotide.

As used herein, the term “planar sample” refers to a substantially flat, i.e., two-dimensional, material (e.g. glass, metal, ceramics, organic polymer surface or gel) that comprises cells or any combination of biomolecules derived from cells, such as proteins, nucleic acids, lipids, oligo/polysaccharides, biomolecule complexes, cellular organelles, cellular debris or excretions (exosomes, microvesicles). A planar cellular sample can be made by, e.g., growing cells on a planar surface, depositing cells on a planar surface, e.g., by centrifugation, by cutting a three dimensional object that contains cells into sections and mounting the sections onto a planar surface, i.e., producing a tissue section, adsorbing the cellular components onto a surface that is functionalized with affinity agents (e.g. antibodies, haptens, nucleic acid probes), introducing the biomolecules into a polymer gel or transferring them onto a polymer surface electrophoretically or by other means. The cells or biomolecules may be fixed using any number of reagents including formalin, methanol, paraformaldehyde, methanol:acetic acid, glutaraldehyde, bifunctional crosslinkers such as bis(succinimidyl)suberate, bis(succinimidyl)polyethyleneglycol, etc. This definition is intended to cover cellular samples (e.g., tissue sections, etc.), electrophoresis gels and blots thereof, Western blots, dot-blots, ELISAs, antibody microarrays, nucleic acid microarrays, etc.

As used herein, the term “tissue section” refers to a piece of tissue that has been obtained from a subject, fixed, sectioned, and mounted on a planar surface, e.g., a microscope slide.

As used herein, the term “formalin-fixed paraffin embedded (FFPE) tissue section” refers to a piece of tissue, e.g., a biopsy sample that has been obtained from a subject, fixed in formaldehyde (e.g., 3%-5% formaldehyde in phosphate buffered saline) or Bouin solution, embedded in wax, cut into thin sections, and then mounted on a microscope slide.

As used herein, the term “non-planar sample” refers to a sample that is not substantially flat, e.g., a whole or partial organ mount (e.g., of a lymph node, brain, liver, etc.), that has been made transparent by means of a refractive index matching technique such as Clear Lipid-exchanged Acrylamide-hybridized Rigid Imaging-compatible Tissue-hydrogel (CLARITY). See, e.g., Roberts et al., J Vis Exp. 2016; (112): 54025. Clearing agents such as benzyl-alcohol/benzyl benzoate (BABB) or benzyl-ether may also be used to render a specimen transparent.

As used herein, the term “spatially-addressable measurements” refers to a set of values that are each associated with a specific position on a surface. Spatially-addressable measurements can be mapped to a position in a sample and can be used to reconstruct an image, e.g., a two- or three-dimensional image, of the sample.

A “diagnostic marker” is a specific biochemical in the body which has a particular molecular feature that makes it useful for detecting a disease, measuring the progress of disease or the effects of treatment, or for measuring a process of interest.

A “pathoindicative” cell is a cell which, when present in a tissue, indicates that the animal in which the tissue is located (or from which the tissue was obtained) is afflicted with a disease or disorder. By way of example, the presence of one or more breast cells in a lung tissue of an animal is an indication that the animal is afflicted with metastatic breast cancer.

The term “complementary site” is used to refer to an epitope for an antibody or aptamer, or nucleic acid that has a sequence that is complementary to an oligonucleotide probe. Specifically, if the binding agent is an antibody or aptamer, then the complementary site for the binding agent is the epitope in the sample to which the antibody or aptamer binds. An epitope may be a conformational epitope or it may be a linear epitope composed of, e.g., a sequence of amino acids. If the binding agent is an oligonucleotide probe, then the complementary site for the binding agent is a complementary nucleic acid (e.g., an RNA or region in a genome).

The term “epitope” as used herein is defined as a structure, e.g., a string of amino acids, on an antigen molecule that is bound by an antibody or aptamer. An antigen can have one or more epitopes. In many cases, an epitope is roughly five amino acids or sugars in size. One skilled in the art understands that generally the overall three-dimensional structure or the specific linear sequence of the molecule can be the main criterion of antigenic specificity.

A “subject” of diagnosis or treatment is a plant or animal, including a human. Non-human animals subject to diagnosis or treatment include, for example, livestock and pets.

As used herein, the term “incubating” refers to maintaining a sample and binding agent under conditions (which conditions include a period of time, one or more temperatures, an appropriate binding buffer and a wash) that are suitable for specific binding of the binding agent to molecules (e.g., epitopes or complementary nucleic acids) in the sample.

As used herein, the term “binding agent” refers to an agent that can specifically bind to complementary sites in a sample. Exemplary binding agents include oligonucleotide probes, antibodies and aptamers. If antibodies or aptamers are used, in many cases they may bind to protein epitopes.

As used herein, the term “binding agent that is linked to a oligonucleotide” refers to a binding agent, e.g., an antibody, aptamer or oligonucleotide probe, that is non-covalently (e.g., via a streptavidin/biotin interaction) or covalently (e.g., via a “click” reaction (see, e.g., Evans Aus. J. Chem. 2007 60: 384-395) or the like) linked to a single-stranded oligonucleotide in a way that the binding agent can still bind to its binding site. The nucleic acid and the binding agent may be linked via a number of different methods, including those that use a cysteine-reactive maleimide or halogen-containing group. The binding agent and the oligonucleotide may be linked proximal to or at the 5′ end of the oligonucleotide, proximal to or at the 3′ end of the oligonucleotide, or anywhere in-between.

The terms “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides, ribonucleotides or a combination thereof, and may be produced enzymatically or synthetically (e.g., PNA as described in U.S. Pat. No. 5,948,902 and the references cited therein) and which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine, thymine, uracil (G, C, A, T and U respectively). DNA and RNA have a deoxyribose and ribose sugar backbone, respectively, whereas PNAs backbone is composed of repeating N-(2-aminoethyl)-glycine units linked by peptide bonds. In PNAs, various purine and pyrimidine bases are linked to the backbone by methylene carbonyl bonds. A locked nucleic acid (LNA), often referred to as an inaccessible RNA, is an RNA molecule comprising modified RNA nucleotides. The ribose moiety of an LNA nucleotide is modified with an extra bridge connecting the 2′ oxygen and 4′ carbon. The bridge “locks” the ribose in the 3′-endo (North) conformation, which is often found in A-form duplexes. LNA nucleotides can be mixed with DNA or RNA residues in the oligonucleotide whenever desired. The term “unstructured nucleic acid”, or “UNA”, is a nucleic acid containing non-natural nucleotides that bind to each other with reduced stability. For example, an unstructured nucleic acid may contain a G′ residue and a C′ residue, where these residues correspond to non-naturally occurring forms, i.e., analogs, of G and C that base pair with each other with reduced stability, but retain an ability to base pair with naturally occurring C and G residues, respectively. Unstructured nucleic acid is described in US20050233340, which is incorporated by reference herein for disclosure of UNA.

As used herein, the term “oligonucleotide” refers to a multimer of at least 10, e.g., at least 15 or at least 30 nucleotides. In some embodiments, an oligonucleotide may be in the range of 15-200 nucleotides in length, or more. Any oligonucleotide used herein may be composed of G, A, T and C, or bases that are capable of base pairing reliably with a complementary nucleotide. 7-deaza-adenine, 7-deaza-guanine, adenine, guanine, cytosine, thymine, uracil, 2-deaza-2-thio-guanosine, 2-thio-7-deaza-guanosine, 2-thio-adenine, 2-thio-7-deaza-adenine, isoguanine, 7-deaza-guanine, 5,6-dihydrouridine, 5,6-dihydrothymine, xanthine, 7-deaza-xanthine, hypoxanthine, 7-deaza-xanthine, 2,6 diamino-7-deaza purine, 5-methyl-cytosine, 5-propynyl-uridine, 5-propynyl-cytidine, 2-thio-thymine or 2-thio-uridine are examples of such bases, although many others are known. As noted above, an oligonucleotide may be an LNA, a PNA, a UNA, or a morpholino oligomer, for example. The oligonucleotides used herein may contain natural or non-natural nucleotides or linkages.

As used herein, the term “reading” in the context of reading a fluorescent signal, refers to obtaining an image by scanning or by microscopy, where the image shows the pattern of fluorescence as well as the intensity of fluorescence in a field of view. The term “reading” also encompasses mass spectrometry methods, e.g., multiplexed ion beam imaging (MIBI) and mass cytometry (CyTOF), as well as other types of microscopy (e.g., SEM).

As used herein, the term “signal generated by”, in the context of, e.g., reading a fluorescent signal generated by addition of the fluorescent nucleotide, refers to a signal that is emitted directly from the fluorescent nucleotide or a signal that is emitted indirectly via energy transfer to another fluorescent nucleotide (i.e., by fluorescence resonance energy transfer (FRET)).

As used herein, the term “activated tyramide” refers to a reactive form of tyramide that has a radical on the C2 position. Non-activated tyramide can be activated by a variety of enzymes (e.g., peroxidase) in the presence of hydrogen peroxide (H₂O₂). In this reaction the phenolic part of tyramide is converted to a short-lived quinone-like structure bearing a radical on the C2 position. Activated tyramide covalently binds to nucleophilic residues (e.g., tyrosines) in close proximity to the reaction (see, e.g., Bobrow et al. J. Immunol. Methods 1992 137:103-112; Bobrow et al. J. Immunol Methods 1989 125:279-285; Van Gijlswijk et al. J. Histochem. Cytochem. 1996 44:389-392; and U.S. Pat. No. 5,196,306).

As used herein, the term “tyramide-label conjugate” refers to a molecule containing a tyramide and a label, where the label is joined to the tyramide via the amide of the tyramide.

As used herein, the term “cleavable linker” refers to a linker containing a bond that can be selectively cleaved by a specific stimulus, e.g., a reducing agent.

Other definitions of terms may appear throughout the specification.

### DETAILED DESCRIPTION

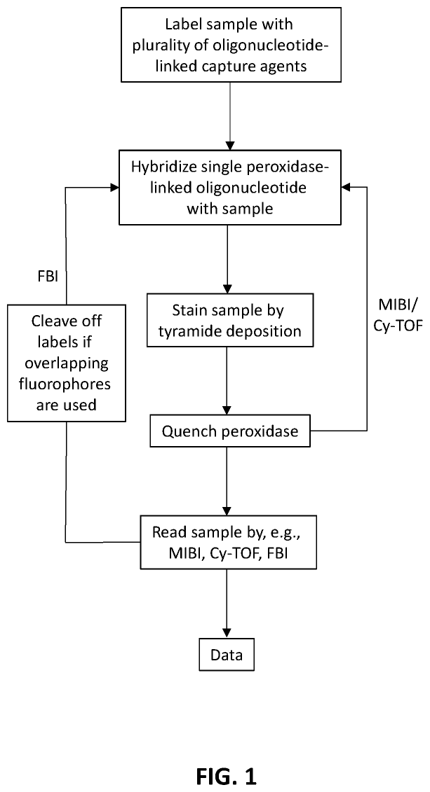

A method for analyzing a sample is provided. Some of the principles of the method are shown in FIG. 1. In some embodiments, the method comprises the steps of obtaining: i. a plurality of binding agents that are each linked to a different oligonucleotide; and ii. a corresponding plurality of peroxidase (e.g., HRP)-linked oligonucleotides (where the term “corresponding” is intended to mean that the number of labeled nucleic acid probes is the same as the number of binding agents used), wherein each of the peroxidase-linked oligonucleotides specifically hybridizes with only one of the binding agents-linked oligonucleotides. For example, if there are 50 binding agents, then they are each linked to a different oligonucleotide and there are 50 peroxidase-linked oligonucleotides, where each oligonucleotide in the peroxidase-linked oligonucleotides is complementary to and specifically hybridizes with only one of the oligonucleotides that are attached to the binding agents. The number of binding agents and peroxidase-linked oligonucleotides used in the method may vary. In some embodiments, the method may be performed using at least 10 or at least 20 binding agents, up to 50 or up 100 or more binding agents, each linked to a different oligonucleotide, and a corresponding number of peroxidase-linked oligonucleotides.

The sequences of the oligonucleotides that are linked to the binding agents may be selected so that they are “orthogonal”, i.e., so that they do not cross-hybridize to one another. Likewise, the sequences of the peroxidase-linked oligonucleotides may be selected so that they are orthogonal and do not cross-hybridize to one another. In addition, the sequences of the oligonucleotides should be designed to minimize binding to other nucleic acids endogenous to the sample (e.g., RNA or DNA).

In some embodiments, the oligonucleotides used in the method may be, independently, 8 nucleotides in length to as long as 150 nucleotides in length (e.g., in the range of 8 to 100 nucleotides in length). However, in many embodiments the oligonucleotides are 8 to 50 nucleotides in length, e.g., 10 to 30 nucleotides or 11 to 25 nucleotides in length although oligonucleotides having a length outside of these ranges can be used in many cases. In some embodiments, an oligonucleotide may have a calculated Tₘ in the range of 15° C. to 70° C. (e.g., 20° C.-60° C. or 35° C.-50° C.). In some embodiments, the oligonucleotides that are linked to the peroxidase may be Tₘ-matched, where the term “Tₘ-matched” refers to sequences that have melting temperatures that are within a defined range, e.g., within less than 15° C., less than 10° C. or less than 5° C. of a defined temperature. Tₘ matching allows the hybridization steps to be performed under the same conditions in each cycle. In some embodiments, the sequences of the oligonucleotides to which the binding agents are linked are the same length and are perfectly complementary to a single peroxidase-linked oligonucleotide.

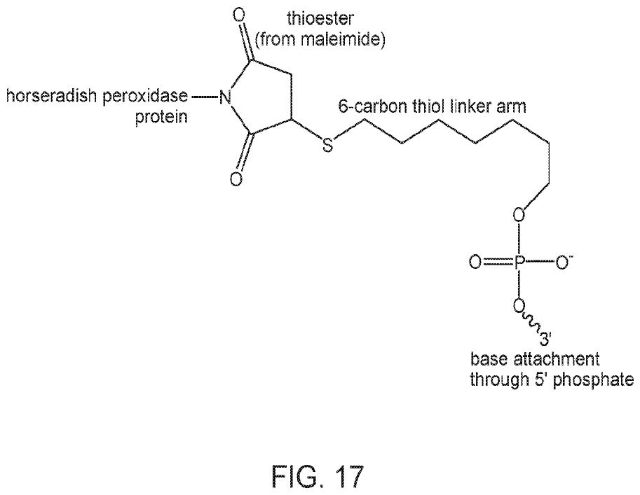

Oligonucleotides may be linked to binding agents or peroxidase using any convenient method (see, e.g., Gong et al., Bioconjugate Chem. 2016 27: 217-225 and Kazane et al. Proc Natl Acad Sci 2012 109: 3731-3736). For example, the unique oligonucleotides may be linked to the binding agents directly using any suitable chemical moiety on the binding agents or peroxidase (e.g., a cysteine residue or via an engineered site). In some embodiments, an oligonucleotide may be linked to the binding agents directly or indirectly via a non-covalent interaction. In some embodiments, the binding agents and the peroxidase may be linked to their respective oligonucleotides by reacting an oligonucleotide-maleimide conjugate with the binding agent or peroxidase, thereby joining those molecules together. An example of such a product is shown in FIG. 17.

In some embodiments, the method may comprise labeling the sample with the plurality of binding agents. This step may involve contacting the sample (e.g., an FFPE section mounted on a planar surface such as a microscope slide) with all of the binding agents, en masse under conditions by which the binding agents bind to complementary sites (e.g., protein epitopes or nucleotide sequences) in the sample. Methods for binding antibodies and aptamers to complementary sites in the sample and methods for hybridizing nucleic acids probes to a sample in situ are well known. In some embodiments, the binding agents may be cross-linked to the sample, thereby preventing the binding agents from disassociating during subsequent steps. This crosslinking step may be done using any amine-to-amine crosslinker although a variety of other chemistries can be used to cross-link the binding agents to the sample if desired. In some embodiments, the binding agents are not cross-linked to the sample.

After the sample has been bound to the binding agents, in some embodiments, the method further comprises specifically hybridizing a single peroxidase-linked oligonucleotide of the plurality of peroxidase-linked oligonucleotides with the sample, thereby producing complexes that comprise the peroxidase. As such, in some embodiments, the method further comprises specifically hybridizing one of the peroxidase-linked oligonucleotides with the binding agent-labeled sample, thereby producing peroxidase-containing complexes that are bound to specific sites in the sample.

After the sample has been washed to remove peroxidase-linked oligonucleotides that have not hybridized to the sample, the method further comprises treating the sample with at least one tyramide-label conjugate (e.g., one, two or three or more tyramide mass-tag conjugates, tyramide-fluorophore conjugates or tyramide-heavy metal conjugates) in the presence of hydrogen peroxide (e.g., about 1 mM H₂O₂). In this step, the peroxidase in the complexes produced in the earlier step activates the tyramide in the conjugate and causes covalent binding of the label to the sample near the complexes. This reaction is similar to the tyramide signal amplification reaction described in Bobrow et al. (J. Immunol. Methods 1992 137:103-112), Bobrow et al. (J. Immunol Methods 1989 125:279-285) and Van Gijlswijk et al. (J. Histochem. Cytochem. 1996 44:389-392) and results in deposition of the label at sites in the sample that are proximal to the binding complex.

After unreacted tyramide-label conjugate has been washed away, the peroxidase can be removed by denaturation or otherwise inactivated (e.g., by treatment with 3-30% hydrogen peroxide w/v for 1 min to 1 hr; see, e.g., Sennepin et al. Analytical Biochemistry 2009 393: 129-131 and Arnao et al. Biochimica et Biophysica Acta (BBA)—Protein Structure and Molecular Enzymology 1990 1038: 85-89) prior to reading the sample to obtain data on the binding of the label.

As shown in FIG. 1, the method may be implemented in a variety of different ways depending on how the sample is going to be read.

In embodiments in which the reading is done by mass spectrometry (e.g., multiplexed ion beam imaging (MIBI) or mass cytometry (CyTOF)), the tyramide-label conjugate used in the method may be a tyramide-mass tag conjugate (which, in many embodiments, is a mass-tag/chelator-tyramide conjugate complex, as shown in FIG. 5). In these embodiments, the term “mass tag” refers to an isotope of any element, including transition metals, post-transition metals, halides, noble metals or lanthanides, that is identifiable by its mass, distinguishable from other mass tags, and used to tag a biologically active material or analyte. A mass tag has an atomic mass that is distinguishable from the atomic masses present in the analytical sample and in the particle of interest. The term “monoisotopic” means that a tag contains a single type of metal isotope (although any one tag may contain multiple metal atoms of the same type). Lanthanides are elements having atomic numbers 58 to 71 and can be readily used herein because they can be chelated by diethylene triamine penta-acetic acid (DTPA) or 1,4,7,10-tetraazacyclododecane-1,4,7,10-tetraacetic acid (DOTA).

In some embodiments, after inactivating the peroxidase, before reading the sample and without removing or inactivating the label, the method further comprises repeating the hybridizing, treating and inactivating steps multiple times, each time using a different peroxidase-linked oligonucleotide of the plurality (i.e., a peroxidase-linked oligonucleotide that has a sequence that is different from the peroxidase-linked oligonucleotides used in the previous steps) and a different tyramide-mass tag conjugate (i.e., a tyramide-mass tag conjugate that contains a mass tag that is distinguishable from the mass tags used in the previous steps). In some embodiments, these steps may be repeated at least 2, at least 5, at least 10 or at least 50 (e.g., 5 to 100) times to produce a sample that is labeled with multiple mass tags. As there are more than 80 naturally occurring elements having more than 250 stable isotopes, the cells may be labeled using at least 5, at least 10, at least 20, at least 30, at least 50, or at least 100, up to 150 mass tags, or more if the mass tags are combined in at least some of the cycles.

In some embodiments, the method further comprises a step of reading the sample by multiplexed ion beam imaging (MIBI). This embodiment method may involve scanning the sample by secondary ion mass spectrometry (SIMS) using a positively or negatively charged ion beam to generate a data set that comprises spatially-addressed measurements of the identity and abundance of the mass tags across the sample. Because ionization removes a layer from the top of the sample and the ion beam can raster through the sample several times, the spatially-addressed measurements can be used to reconstruct a two-dimensional or three-dimensional image of the sample. The general principles of MIBI, including methods by which samples may be made, methods for ionizing the tags, and methods for analyzing the data, as well as hardware that can be employed in MIBI, including but not limited to, mass spectrometers and computer control systems are known and are reviewed in a variety of publications including, but not limited to Angelo et al. Nature Medicine 2014 20:436, Rost et al. Lab. Invest. 2017 97: 992-1003, U.S. Pat. Nos. 9,766,224, 9,312,111 and US2015/0080233, among many others, which patents and publications are incorporated by reference herein for disclosure of those methods and hardware.

Alternatively, in some embodiments, the method further comprises a step of reading the sample by mass cytometry (CyTOF). In these embodiments, the sample may comprise a suspension of disassociated cells that are separated from another and capable of being sorted in a flow cytometer. As such, in these embodiments, the cells may be labeled in solution and washed after each step. In these embodiments, the population of cells may be obtained from blood (e.g., peripheral blood mononuclear cells (PBMC) such as lymphocytes, monocytes, macrophages, etc., red blood cells, neutrophils, eosinophils, basophils, etc., or other cells that are circulating in peripheral blood), cells that are grown in culture such as a suspension of single cells, and single cell organisms. In some cases, the sample may be made from a tissue sample (particularly of a soft tissue such as, e.g., spleen, liver or brain) or cultured cells (e.g., human embryonic kidney cells, COS cells, HeLa cells, Chinese hamster ovary cells, cancer cell lines; stem cell lines, such as embryonic stem cells and induced pluripotent stem cells, etc.) that have been trypsinized to physically disassociate the cells from one another.

Mass cytometry makes uses a plasma beam to atomize mass-tag labeled cells in a sample and generate a data set that comprises temporally-addressable measurements of the abundance of the mass tags in or on each of the analyzed cells. In mass cytometry, mass-tag labeled cells are introduced into a fluidic system and hydrodynamically focused one cell at a time through a flow cell using a sheath fluid prior to being vaporized, atomized and ionized by plasma (e.g., an inductively coupled plasma) to produce ions that are subsequently analyzed by spectrometry (using, e.g., a mass spectrometer or an emission spectrometer) to determine the identity and/or relative abundance of the mass tags associated with the cell. The general principles of mass cytometry, including methods by which single cell suspensions can be made, methods by which cells can be labeled using, e.g., mass-tagged antibodies, methods for atomizing particles and methods for performing elemental analysis on particles, as well as hardware that can be employed in mass cytometry, including flow cells, ionization chambers, reagents, mass spectrometers and computer control systems are well-known and have been amply reviewed in a variety of publications including, but not limited to Bandura et al. Analytical Chemistry 2009 81: 6813-6822), Tanner et al. (Pure Appl. Chem 2008 80: 2627-2641), U.S. Pat. No. 7,479,630 (Method and apparatus for flow cytometry linked with elemental analysis) and U.S. Pat. No. 7,135,296 (Elemental analysis of tagged biologically active materials); and published U.S. patent application 20080046194, for example, which publications are incorporated by reference herein for disclosure of those methods and hardware.

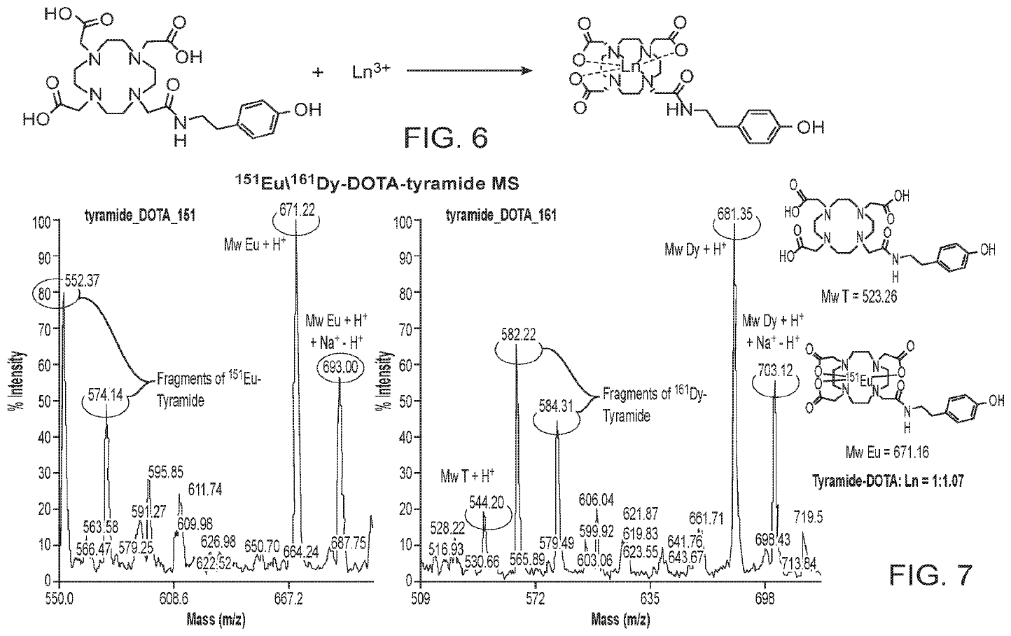

In some embodiments, the tyramide-mass tag conjugates may be composed of a tyramide-chelator conjugate and a stable metal isotope that is bound by the chelator, as illustrated in FIG. 6. The chelator may be, e.g., DTPA or DOTA. The stable metal isotope used in the method may be any stable isotope that is not commonly found in the sample under analysis. These may include, but are not limited to, the high molecular weight members of the transition metals (e.g., Rh, Ir, Cd, Au), post-transition metals (e.g., Al, Ga, In, Tl), metalloids (e.g., Te, Bi), alkaline metals, halogens, and actinides, although others may be used in some circumstances. A mass tag may have an atomic number in the range of 21 to 238. In certain embodiments, a lanthanide may be used. The lanthanide series of the periodic table comprises 15 elements, 14 of which have stable isotopes (La, Ce, Pr, Nd, Sm, Eu, Gd, Tb, Dy, Ho, Er, Tm, Yb, Lu). Lanthanides can be readily used because of their rarity in the biosphere. There are more than 100 stable isotopes of elements having an atomic number between 1 and 238 that are not commonly found in biological systems. In some embodiments, tagging isotopes may comprise non-lanthanide elements that can form stable metal chelator tags for the applications described herein. When using a SIMS-based measurement method, in contrast to some inductively coupled plasma mass spectrometry (ICP-MS)-based methods, the elemental reporter could also comprise lower MW transition elements not common in biological systems (e.g. Al, W, and Hg). Elements suitable for use in this method in certain embodiments include, but are not limited to, lanthanides and noble metals such as gold, silver or platinum. In certain cases, an elemental tag may have an atomic number of 21-92. In particular embodiments, the elemental tag may contain a transition metal, i.e., an element having the following atomic numbers, 21-29, 39-47, 57-79, and 89. Transition elements include the lanthanides and noble metals. See, e.g., Cotton and Wilkinson, 1972, pages 528-530. The elemental tags employed herein are not commonly present in typical biological samples, e.g., cells, unless they are provided exogenously.

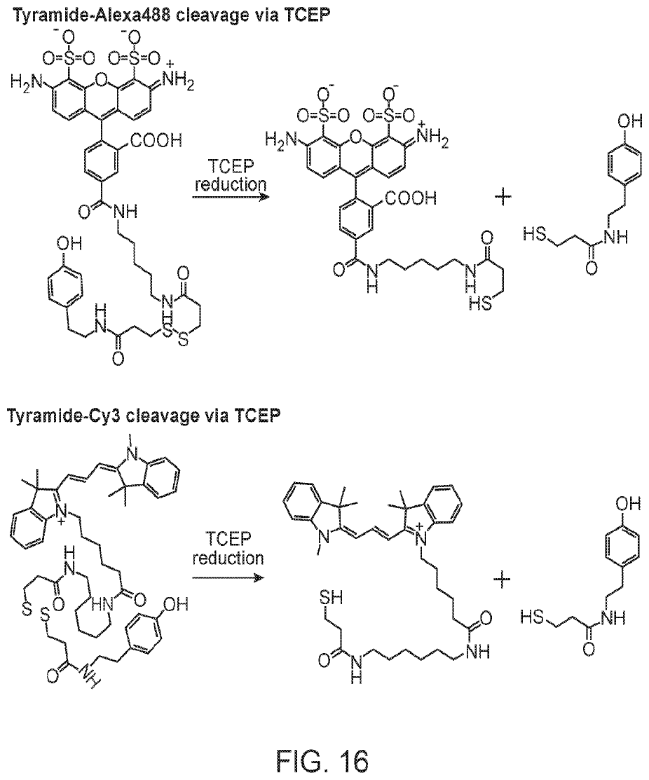

In certain embodiments, the reading is done by fluorescence-based imaging (FBI) and the tyramide-label conjugate may be a tyramide-fluorophore conjugate, two examples of which are illustrated in FIG. 16. Fluorophores of interest include but are not limited to xanthene dyes, e.g., fluorescein and rhodamine dyes, such as fluorescein isothiocyanate (FITC), 6-carboxyfluorescein (commonly known by the abbreviations FAM and F), 6-carboxy-2′,4′,7′,4,7-hexachlorofluorescein (HEX), 6-carboxy-4′, 5′-dichloro-2′, 7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G⁵ or G⁵), 6-carboxyrhodamine-6G (R6G⁶ or G⁶), and rhodamine 110; cyanine dyes, e.g., Cy3, Cy5 and Cy7 dyes; coumarins, e.g., umbelliferone; benzimide dyes, e.g. Hoechst 33258; phenanthridine dyes, e.g., Texas Red; ethidium dyes; acridine dyes; carbazole dyes; phenoxazine dyes; porphyrin dyes; polymethine dyes, e.g., BODIPY dyes and quinoline dyes.

In some embodiments, the reading is done by fluorescence-based imaging (FBI) to detect samples labeled with two, three, or four distinguishable fluorophores and the method comprises repeating the hybridization, treatment and inactivation steps multiple times (at least one or twice, up to the number of distinguishable fluorophores), each time using a different peroxidase-linked oligonucleotide and a different tyramide-fluorophore conjugate, prior to reading the sample by fluorescence microscopy to produce an image showing the pattern of binding of the label to the sample. In some embodiments, the tyramide-label conjugate may be a tyramide-fluorophore conjugate that comprises a selectively cleavable linker disposed between the tyramide and the fluorophore (as shown in FIG. 16). In some embodiments, after the reading step, the method may involve chemically removing the label that is associated with the sample by cleaving the cleavable linker, thereby leaving the plurality of binding agents and their associated oligonucleotides still bound to the sample, and repeating the hybridization, treatment, inactivation and reading steps multiple times (e.g., 5 to 100 times), each time using a different peroxidase-linked oligonucleotide and followed by the cleavage step except for the final repeat, to produce a plurality of images of the sample, each image corresponding to a particular peroxidase-linked oligonucleotide of the plurality of peroxidase-linked oligonucleotides used in the method. In these embodiments, the method may comprise reading the sample to obtain an image showing the binding pattern for the particular peroxide-linked oligonucleotide hybridized, treated, and inactivated in the prior step. This step may be done using any convenient reading method and, in some embodiments, e.g., hybridization of the different probes can be separately read using a fluorescence microscope equipped with an appropriate filter for the fluorophore used, or by using dual or triple band-pass filter sets to observe multiple fluorophores (see, e.g., U.S. Pat. No. 5,776,688), as appropriate.

If the tyramide-fluorophore conjugate contains a cleavable linker, then the cleavable linker should be capable of being selectively cleaved using a stimulus (e.g., a chemical, light or a change in its environment) without breaking any bonds in the oligonucleotides. In some embodiments, the cleavable linkage may be a disulfide bond, which can be readily broken using a reducing agent (e.g., β-mercaptoethanol, TCEP or the like). Suitable cleavable bonds that may be employed include, but are not limited to, the following: base-cleavable sites such as esters, particularly succinates (cleavable by, for example, ammonia or trimethylamine), quaternary ammonium salts (cleavable by, for example, diisopropylamine) and urethanes (cleavable by aqueous sodium hydroxide); acid-cleavable sites such as benzyl alcohol derivatives (cleavable using trifluoroacetic acid), teicoplanin aglycone (cleavable by trifluoroacetic acid followed by base), acetals and thioacetals (also cleavable by trifluoroacetic acid), thioethers (cleavable, for example, by HF or cresol) and sulfonyls (cleavable by trifluoromethane sulfonic acid, trifluoroacetic acid, thioanisole, or the like); nucleophile-cleavable sites such as phthalamide (cleavable by substituted hydrazines), esters (cleavable by, for example, aluminum trichloride); and Weinreb amide (cleavable by lithium aluminum hydride); and other types of chemically cleavable sites, including phosphorothioate (cleavable by silver or mercuric ions) and diisopropyldialkoxysilyl (cleavable by fluoride ions). Other cleavable bonds will be apparent to those skilled in the art or are described in the pertinent literature and texts (e.g., Brown (1997) Contemporary Organic Synthesis 4(3); 216-237). In some embodiments, a cleavable bond may be cleaved by an enzyme. In particular embodiments, a photocleavable (“PC”) linker (e.g., a uv-cleavable linker) may be employed. Suitable photocleavable linkers for use may include ortho-nitrobenzyl-based linkers, phenacyl linkers, alkoxybenzoin linkers, chromium arene complex linkers, NpSSMpact linkers and pivaloylglycol linkers, as described in Guillier et al. (Chem Rev. 2000 Jun. 14; 100(6):2091-158). Exemplary linking groups that may be employed in the subject methods may be described in Guillier et al., supra and Olejnik et al. (Methods in Enzymology 1998 291:135-154), and further described in U.S. Pat. No. 6,027,890; Olejnik et al. (Proc. Natl. Acad Sci, 92:7590-94); Ogata et al. (Anal. Chem. 2002 74:4702-4708); Bai et al. (Nucl. Acids Res. 2004 32:535-541); Zhao et al. (Anal. Chem. 2002 74:4259-4268); and Sanford et al. (Chem Mater. 1998 10:1510-20), and are purchasable from Ambergen (Boston, Mass.; NHS-PC-LC-Biotin), Link Technologies (Bellshill, Scotland), Fisher Scientific (Pittsburgh, Pa.) and Calbiochem-Novabiochem Corp. (La Jolla, Calif.).

In some embodiments (and as shown in FIG. 16) the cleavable linker may comprise a linkage cleavable by a reducing agent (e.g., a disulfide bond). In these embodiments, the label may be removed using a reducing agent, e.g., tris(2-carboxyethyl)phosphine (TCEP).

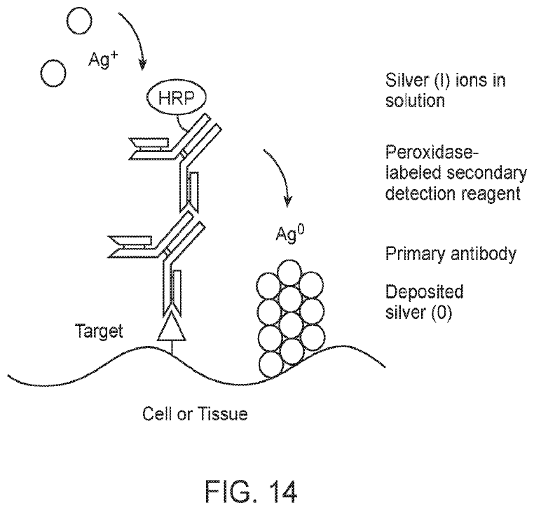

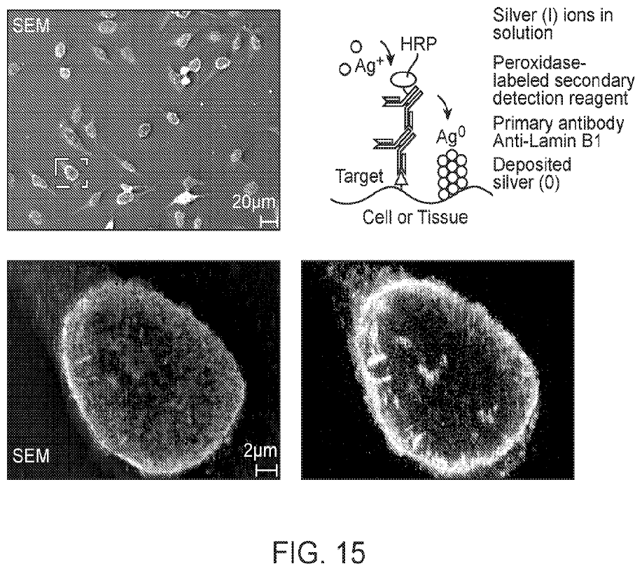

In some embodiments, the tyramide-label conjugate may comprise a heavy metal (e.g., lead, gold, palladium, platinum, or uranium, etc.) and the reading step may be done by electron microscopy (e.g., scanning electron microscopy), as illustrated in FIGS. 14 and 15.

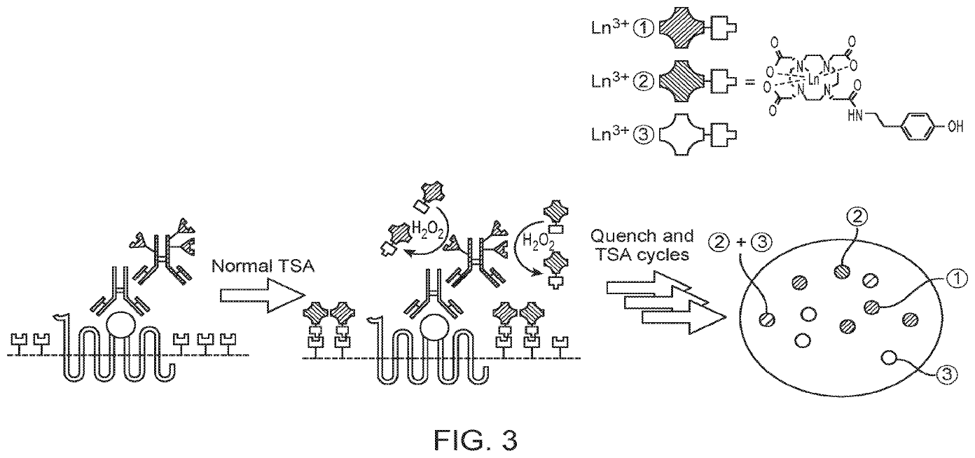

In some embodiments, the sample may be treated with a single tyramide-label conjugate in each cycle, thereby labeling the sample with a single label in each cycle. In other embodiments, the sample may treated with multiple (e.g., up to two, three, four or five) distinguishable tyramide-label conjugates in each cycle, thereby labeling the sample with multiple labels in each cycle. These latter embodiments (as illustrated in FIG. 3) can be used to increase the level of multiplexing since sites that are labeled with a particular combination of labels are distinguishable from sites that are labeled with a single label or other combinations of labels.

In embodiments in which the sample is read by multiplexed ion beam imaging (MIBI), each reading step may produce an image of the sample showing the pattern of binding of multiple binding agents. In particular embodiments, in any one pixel of the image, the intensity of the color of the pixel correlates with the magnitude of the signals obtained for a mass tag obtained in the original scanning. In these embodiments, the resulting false color image may show color-code cells in which the intensity of the color in any single pixel of a cell correlates with the amount of specific binding reagent that is associated with the corresponding area in the sample.

In embodiments in which the sample is read by fluorescence, each reading step may produce an image of the sample showing the pattern of binding of a single binding agent. In some embodiments, the method may further comprise analyzing, comparing or overlaying, at least two of the images. In some embodiments, the method may further comprise overlaying all of the images to produce an image showing the pattern of binding of all of the binding agents to the sample. The image analysis module used may transform the signals from each fluorophore to produce a plurality of false color images. The image analysis module may overlay the plurality of false color images (e.g., superimposing the false colors at each pixel) to obtain a multiplexed false color image. Multiple images (e.g., unweighted or weighted) may be transformed into a single false color, e.g., so as to represent a biological feature of interest characterized by the binding of specific binding agent. False colors may be assigned to specific binding agents or combinations of binding agents, based on manual input from the user. In certain aspects, the image may comprise false colors relating only to the intensities of labels associated with a feature of interest, such as in the nuclear compartment. The image analysis module may further be configured to adjust (e.g., normalize) the intensity and/or contrast of signal intensities or false colors, to perform a deconvolution operation (such as blurring or sharpening of the intensities or false colors), or perform any other suitable operations to enhance the image. The image analysis module may perform any of the above operations to align pixels obtained from successive images and/or to blur or smooth intensities or false colors across pixels obtained from successive images.

In some embodiments, images of the sample may be taken at different focal planes, in the z direction. These optical sections can be used to reconstruct a three dimensional image of the sample. Optical sections may be taken using confocal microscopy, or by any other method known to an artisan of ordinary skill in the biological arts.

In addition to the labeling methods described above, the sample may be stained using a cytological stain, either before or after performing the method described above. In these embodiments, the stain may be, for example, phalloidin, gadodiamide, acridine orange, bismarck brown, barmine, Coomassie blue, bresyl violet, brystal violet, DAPI, hematoxylin, eosin, ethidium bromide, acid fuchsine, haematoxylin, hoechst stains, iodine, malachite green, methyl green, methylene blue, neutral red, Nile blue, Nile red, osmium tetroxide (formal name: osmium tetraoxide), rhodamine, safranin, phosphotungstic acid, osmium tetroxide, ruthenium tetroxide, ammonium molybdate, cadmium iodide, carbohydrazide, ferric chloride, hexamine, indium trichloride, lanthanum nitrate, lead acetate, lead citrate, lead(II) nitrate, periodic acid, phosphomolybdic acid, potassium ferricyanide, potassium ferrocyanide, ruthenium red, silver nitrate, silver proteinate, sodium chloroaurate, thallium nitrate, thiosemicarbazide, uranyl acetate, uranyl nitrate, vanadyl sulfate, or any derivative thereof. The stain may be specific for any feature of interest, such as a protein or class of proteins, phospholipids, DNA (e.g., dsDNA, ssDNA), RNA, an organelle (e.g., cell membrane, mitochondria, endoplasmic recticulum, golgi body, nuclear envelope, and so forth), or a compartment of the cell (e.g., cytosol, nuclear fraction, and so forth). The stain may enhance contrast or imaging of intracellular or extracellular structures. In some embodiments, the sample may be stained with haematoxylin and eosin (H&E).

### Other Embodiments

Also provided is a labeling system comprising: a plurality of (e.g., up to 5, 10, 20, 25, 50, 75, 100, or more) binding agents that are each linked to a different oligonucleotide; a corresponding plurality of peroxidase-linked oligonucleotides, wherein each of the peroxidase-linked oligonucleotides specifically hybridizes with only one of the oligonucleotides, and a tyramide-label conjugate, wherein the tyramide of the conjugate is activatable by peroxidase treatment. As described above, the tyramide-label conjugate may comprise a mass tag, a heavy metal or a fluorophore. In embodiments in which the label is a fluorophore, the tyramide and label of the tyramide-label conjugate may be joined by a cleavable linker, as described above.

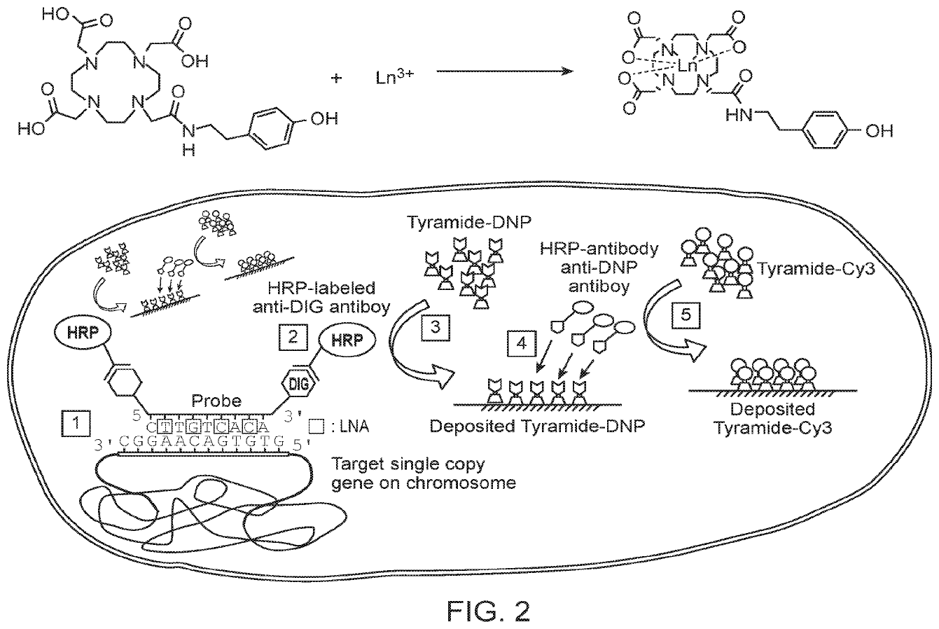

Also provided is a reagent system comprising: tyramide linked to a metal chelator (e.g., DTPA or DOTA) or a heavy metal, as illustrated by example in FIG. 2. In these embodiments, the reagent system may comprise a peroxidase-linked binding agent, as illustrated in FIGS. 14 and 15. In these embodiments, the system may further comprise a mass tag, e.g., a lanthanide, that is either separate or in a complex with the chelator.

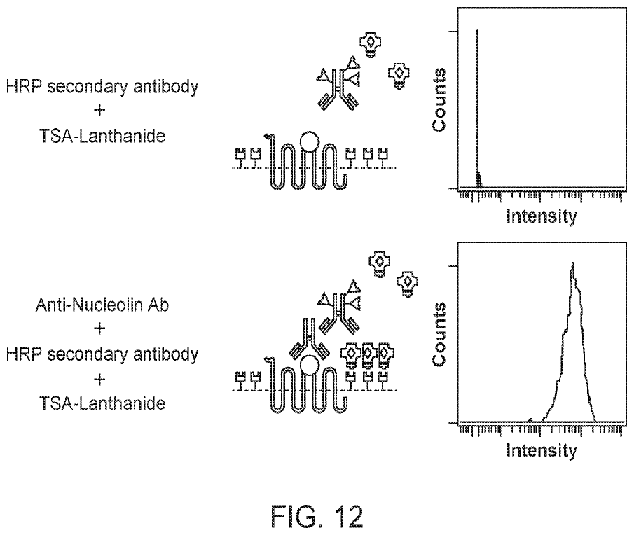

Also provided is a method for analyzing a sample. In some embodiments, this method may comprise: labeling a sample with a peroxidase-linked binding agent, treating the sample with a mass-tag/chelator-tyramide conjugate complex, wherein the peroxidase of the capture binding bound to the sample in (a) activates the conjugate and causes covalent binding of the label to the sample near the sites to which the binding agent has bound; and reading the sample, e.g., by a mass spectrometry-based method capable of detecting mass tags (e.g., by MIBI, mass cytometry) or electron microscopy. This embodiment is illustrated in FIG. 12.

### Kits

Also provided by this disclosure are kits that contain reagents for practicing the subject methods, as described above. These various components of a kit may be in separate vessels or mixed in the same vessel.

The various components of the kit may be present in separate containers or certain compatible components may be pre-combined into a single container, as desired.

In addition to the above-mentioned components, the subject kit may further include instructions for using the components of the kit to practice the subject method.

### Utility

The methods and compositions described herein find general use in a wide variety of applications for analysis of any sample (e.g., in the analysis of tissue sections, sheets of cells, spun-down cells, cell suspensions, blots of electrophoresis gels, Western blots, dot-blots, ELISAs, antibody microarrays, nucleic acid microarrays, whole tissues or parts thereof, or non-planar pieces of tissue etc.). The method may be used to analyze any tissue, including tissue that has been clarified, e.g., through lipid elimination, for example. The sample may be prepared using expansion microscopy methods (see, e.g., Chozinski et al. Nature Methods 2016 13: 485-488), which involves creating polymer replicas of a biological system created through selective co-polymerization of organic polymer and cell components. The method can be used to analyze spreads of cells, exosomes, extracellular structures, biomolecules deposited on a solid support or in a gel (Elisa, western blot, dot blot), whole organism, individual organs, tissues, cells, extracellular components, organelles, cellular components, chromatin and epigenetic markers, biomolecules and biomolecular complexes, for example. The binding agents may bind to any type of molecule, including proteins, lipids, polysaccharides, proteoglycans, metabolites, or artificial small molecules or the like. The method may have many biomedical applications in screening and drug discovery and the like. Further, the method has a variety of clinical applications, including, but not limited to, diagnostics, prognostics, disease stratification, personalized medicine, clinical trials and drug accompanying tests.

In particular embodiments, the sample may be a section of a tissue biopsy obtained from a patient. Biopsies of interest include both tumor and non-neoplastic biopsies of skin (melanomas, carcinomas, etc.), soft tissue, bone, breast, colon, liver, kidney, adrenal, gastrointestinal, pancreatic, gall bladder, salivary gland, cervical, ovary, uterus, testis, prostate, lung, thymus, thyroid, parathyroid, pituitary (adenomas, etc.), brain, spinal cord, ocular, nerve, and skeletal muscle, etc.

In certain embodiments, binding agents specifically bind to biomarkers, including cancer biomarkers, that may be proteinaceous. Exemplary cancer biomarkers, include, but are not limited to carcinoembryonic antigen (for identification of adenocarcinomas), cytokeratins (for identification of carcinomas but may also be expressed in some sarcomas), CD15 and CD30 (for Hodgkin's disease), alpha fetoprotein (for yolk sac tumors and hepatocellular carcinoma), CD117 (for gastrointestinal stromal tumors), CD10 (for renal cell carcinoma and acute lymphoblastic leukemia), prostate specific antigen (for prostate cancer), estrogens and progesterone (for tumour identification), CD20 (for identification of B-cell lymphomas) and CD3 (for identification of T-cell lymphomas).

The above-described method can be used to analyze cells from a subject to determine, for example, whether the cell is normal or not or to determine whether the cells are responding to a treatment. In one embodiment, the method may be employed to determine the degree of dysplasia in cancer cells. In these embodiments, the cells may be a sample from a multicellular organism. A biological sample may be isolated from an individual, e.g., from a soft tissue. In particular cases, the method may be used to distinguish different types of cancer cells in FFPE samples.

The method described above finds particular utility in examining samples using a plurality of antibodies, each antibody recognizing a different marker. Examples of cancers, and biomarkers that can be used to identify those cancers, are shown below. In these embodiments, one does not need to examine all of the markers listed below in order to make a diagnosis.

| | |
|---|---|
| Acute Leukemia IHC Panel | CD3, CD7, CD20, CD34, CD45, CD56, CD117, MPO, PAX-5, |
| | and TdT. |
| Adenocarcinoma vs. Mesothelioma IHC | Pan-CK, CEA, MOC-31, BerEP4, TTF1, calretinin, and WT-1. |
| Panel | |
| Bladder vs. Prostate Carcinoma IHC Panel | CK7, CK20, PSA, CK 903, and p63. |
| Breast IHC Panel | ER, PR, Ki-67, and HER2. Reflex to HER2 FISH after HER2 |
| | IHC is available. |
| Burkitt vs. DLBC Lymphoma IHC panel | BCL-2, c-MYC, Ki-67. |
| Carcinoma Unknown Primary Site, Female | CK7, CK20, mammaglobin, ER, TTF1, CEA, CA19-9, S100, |
| (CUPS IHC Panel - Female) | synaptophysin, and WT-1. |
| Carcinoma Unknown Primary Site, Male | CK7, CK20, TTF1, PSA, CEA, CA19-9, S100, and |
| (CUPS IHC Panel - Male) | synaptophysin. |
| GIST IHC Panel | CD117, DOG-1, CD34, and desmin. |
| Hepatoma/Cholangio vs. Metastatic | HSA (HepPar 1), CDX2, CK7, CK20, CAM 5.2, TTF-1, and |
| Carcinoma IHC Panel | CEA (polyclonal). |
| Hodgkin vs. NHL IHC Panel | BOB-1, BCL-6, CD3, CD10, CD15, CD20, CD30, CD45 LCA, |
| | CD79a, MUM1, OCT-2, PAX-5, and EBER ISH. |
| Lung Cancer IHC Panel | chromogranin A, synaptophysin, CK7, p63, and TTF-1. |
| Lung vs. Metastatic Breast Carcinoma IHC | TTF1, mammaglobin, GCDFP-15 (BRST-2), and ER. |
| Panel | |
| Lymphoma Phenotype IHC Panel | BCL-2, BCL-6, CD3, CD4, CD5, CD7, CD8, CD10, CD15, |
| | CD20, CD30, CD79a, CD138, cyclin D1, Ki67, MUM1, PAX- |
| | 5, TdT, and EBER ISH. |
| Lymphoma vs. Carcinoma IHC Panel | CD30, CD45, CD68, CD117, pan-keratin, MPO, S100, and |
| | synaptophysin. |
| Lymphoma vs. Reactive Hyperplasia IHC | BCL-2, BCL-6, CD3, CD5, CD10, CD20, CD23, CD43, cyclin |
| Panel | D1, and Ki-67. |
| Melanoma vs. Squamous Cell Carcinoma | CD68, Factor XIIIa, CEA (polyclonal), S-100, melanoma |
| IHC Panel | cocktail (HMB-45, MART-1/Melan-A, tyrosinase) and Pan- |
| | CK. |
| Mismatch Repair Proteins IHC Panel | MLH1, MSH2, MSH6, and PMS2. |
| (MMR/Colon Cancer) | |
| Neuroendocrine Neoplasm IHC Panel | CD56, synaptophysin, chromogranin A, TTF-1, Pan-CK, and |
| | CEA (polyclonal). |
| Plasma Cell Neoplasm IHC Panel | CD19, CD20, CD38, CD43, CD56, CD79a, CD138, cyclin D1, |
| | EMA, IgG kappa, IgG lambda, and MUM1. |
| Prostate vs. Colon Carcinoma IHC Panel | CDX2, CK 20, CEA (monoclonal), CA19-9, PLAP, CK 7, and |
| | PSA. |
| Soft Tissue Tumor IHC Panel | Pan-CK, SMA, desmin, S100, CD34, vimentin, and CD68. |
| T-Cell Lymphoma IHC panel | ALK1, CD2, CD3, CD4, CD5, CD7, CD8, CD10, CD20, |
| | CD21, CD30, CD56, TdT, and EBER ISH. |
| T-LGL Leukemia IHC panel | CD3, CD8, granzyme B, and TIA-1. |
| Undifferentiated Tumor IHC Panel | Pan-CK, S100, CD45, and vimentin. |

In some embodiments, the method may involve obtaining data (an image) as described above (an electronic form of which may have been forwarded from a remote location), and the image may be analyzed by a doctor or other medical professional to determine whether a patient has abnormal cells (e.g., cancerous cells) or which type of abnormal cells are present. The image may be used as a diagnostic to determine whether the subject has a disease or condition, e.g., a cancer. In certain embodiments, the method may be used to determine the stage of a cancer, to identify metastasized cells, or to monitor a patient's response to a treatment, for example.

The compositions and methods described herein can be used to diagnose a patient with a disease. In some cases, the presence or absence of a biomarker in the patient's sample can indicate that the patient has a particular disease (e.g., a cancer). In some cases, a patient can be diagnosed with a disease by comparing a sample from the patient with a sample from a healthy control. In this example, a level of a biomarker, relative to the control, can be measured. A difference in the level of a biomarker in the patient's sample relative to the control can be indicative of disease. In some cases, one or more biomarkers are analyzed in order to diagnose a patient with a disease. The compositions and methods of the disclosure are particularly suited for identifying the presence or absence of, or determining expression levels, of a plurality of biomarkers in a sample.

In some cases, the compositions and methods herein can be used to determine a treatment plan for a patient. The presence or absence of a biomarker may indicate that a patient is responsive to or refractory to a particular therapy. For example, a presence or absence of one or more biomarkers may indicate that a disease is refractory to a specific therapy, and an alternative therapy can be administered. In some cases, a patient is currently receiving the therapy and the presence or absence of one or more biomarkers may indicate that the therapy is no longer effective.

In some cases, the method may be employed in a variety of diagnostic, drug discovery, and research applications that include, but are not limited to, diagnosis or monitoring of a disease or condition (where the image identifies a marker for the disease or condition), discovery of drug targets (where the a marker in the image may be targeted for drug therapy), drug screening (where the effects of a drug are monitored by a marker shown in the image), determining drug susceptibility (where drug susceptibility is associated with a marker) and basic research (where is it desirable to measure the differences between cells in a sample).

In certain embodiments, two different samples may be compared using the above methods. The different samples may be composed of an “experimental” sample, i.e., a sample of interest, and a “control” sample to which the experimental sample may be compared. In many embodiments, the different samples are pairs of cell types or fractions thereof, one cell type being a cell type of interest, e.g., an abnormal cell, and the other a control, e.g., normal, cell. If two fractions of cells are compared, the fractions are usually the same fraction from each of the two cells. In certain embodiments, however, two fractions of the same cell may be compared. Exemplary cell type pairs include, for example, cells isolated from a tissue biopsy (e.g., from a tissue having a disease such as colon, breast, prostate, lung, skin cancer, or infected with a pathogen, etc.) and normal cells from the same tissue, usually from the same patient; cells grown in tissue culture that are immortal (e.g., cells with a proliferative mutation or an immortalizing transgene), infected with a pathogen, or treated (e.g., with environmental or chemical agents such as peptides, hormones, altered temperature, growth condition, physical stress, cellular transformation, etc.), and a normal cell (e.g., a cell that is otherwise identical to the experimental cell except that it is not immortal, infected, or treated, etc.); a cell isolated from a mammal with a cancer, a disease, a geriatric mammal, or a mammal exposed to a condition, and a cell from a mammal of the same species, preferably from the same family, that is healthy or young; and differentiated cells and non-differentiated cells from the same mammal (e.g., one cell being the progenitor of the other in a mammal, for example). In one embodiment, cells of different types, e.g., neuronal and non-neuronal cells, or cells of different status (e.g., before and after a stimulus on the cells) may be employed. In another embodiment of the invention, the experimental material contains cells that are susceptible to infection by a pathogen such as a virus, e.g., human immunodeficiency virus (HIV), etc., and the control material contains cells that are resistant to infection by the pathogen. In another embodiment, the sample pair is represented by undifferentiated cells, e.g., stem cells, and differentiated cells.

The images produced by the method may be viewed side-by-side or, in some embodiments, the images may be superimposed or combined. In some cases, the images may be in color, where the colors used in the images may correspond to the labels used.

Cells from any organism, e.g., from bacteria, yeast, plants and animals, such as fish, birds, reptiles, amphibians and mammals may be used in the subject methods. In certain embodiments, mammalian cells, i.e., cells from mice, rabbits, primates, or humans, or cultured derivatives thereof, may be used.

### Examples

In order to further illustrate some embodiments of the present invention, the following specific examples are given with the understanding that they are being offered to illustrate examples of the present invention and should not be construed in any way as limiting its scope.

### TSA Staining

FFPE tissues or cell pellets were cut onto standard microscopy slides (25×75 mm) treated with vectabond per manufacturer protocol (SP-1800, Vector Labs). For MIBI samples, the slides were seeded with a thin layer of tantalum followed by gold (for details, see Keren et al 2018 Cell).

Slides were baked for 1 hour at 70° C., before going through a deparaffinization protocol via sequential dipping for 3 minutes in the following: 3× xylene, 2×100% EtOH, 2×95% EtOH, 1×80% EtOH, 1×70% EtOH and 3× ddH2O. The slides then go through epitope retrieval at 97° C. for 30 minutes in epitope retrieval buffer (322000, ACDBio), followed by a H2O2 blocking for endogenous peroxide activity (0.3% H2O2 in 1×PBS).

Primary oligo probes, anti-sense to the sequence of interest for detection, were hybridized overnight with the sample in hybridization buffer (30% Formamide, 2 uM oligo probes in 2×SSC-T). The slides were then washed twice in 1× RNAscope Wash Buffer (320058, ACDBio) for 5 minutes each time. The slides were then incubated for 1 hour in a secondary detection buffer (0.6 um secondary probe conjugated to HRP, complementary to the primary probe, 30% Formamide in 2×SSC-T). The slides were then washed twice in 1× RNAscope Wash Buffer for 5 minutes each time.

Tyramide reagents are then added to the slides to begin the tyramide reaction (Alexa-488-tyramide for glass slides, and DOTA-lanthanide-tyramide for gold slides). The reactions are stopped after 10 minutes by washing twice in 1× RNAscope wash buffer, and the results checked on a fluorescence microscope (BZ-X, Keyence) or the MIBIscope (Ionpath).

### TSA Staining of Alpha-Satellite Repeats

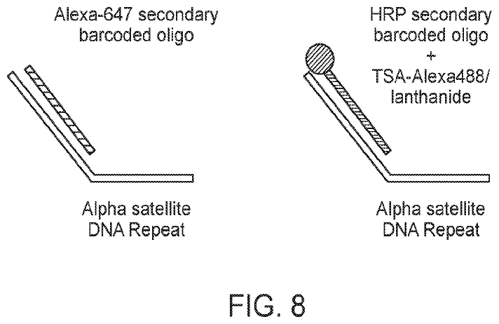

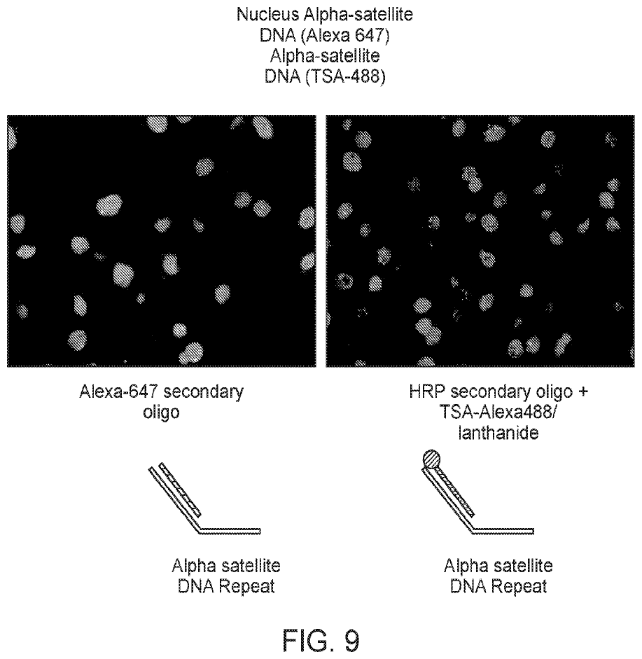

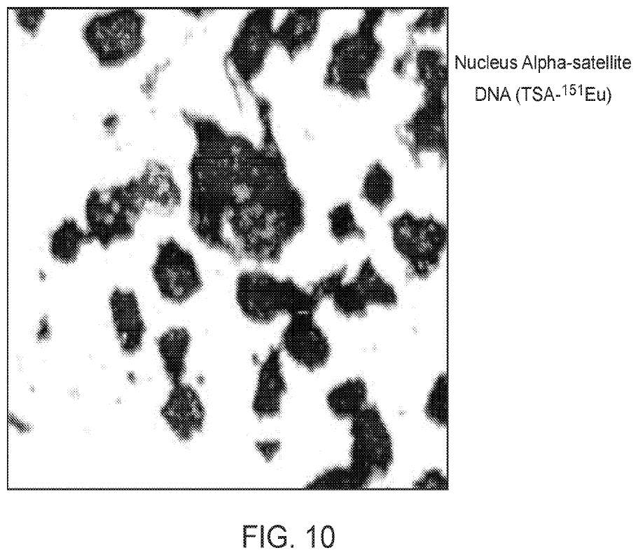

FIG. 8 illustrates the design of two systems for detecting alpha-satellite DNA. Both systems rely on the same binding agent, that i. has a region that hybridizes to the alpha-satellite repeat and ii. has a region that hybridizes to a secondary oligonucleotide. In the first system (the system on the left) the second oligonucleotide is labeled with Alexa Fluor® 647. In the second system (the system on the right) the second oligonucleotide is conjugated to HRP labeled and used in conjunction with a tyramide-Alexa Fluor® 488 conjugate (or a tyramide-mass tag conjugate). The probes were hybridized to cells in situ and read. As can be seen from FIG. 9, both probes stain the same regions in the cell, but the system that uses the HRP/tyramide conjugate provides stronger staining. FIG. 10 shows how the second probe system can be used in conjunction with a tyramide-mass tag (¹⁵¹Eu) conjugate to stain alpha-satellite DNA, as detected by MIBI.

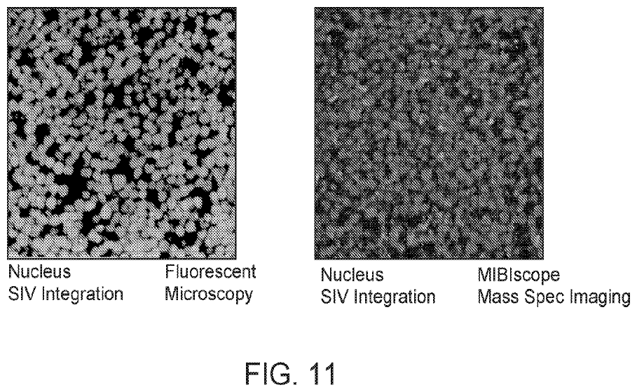

A similar approach was used to detect SIV. FIG. 11 shows the results of these experiments. As shown, cells that are infected with SIV can be detected using fluorescence microscopy or mass spectrometry imaging (MIBI).

### TSA Staining of Nucleolin

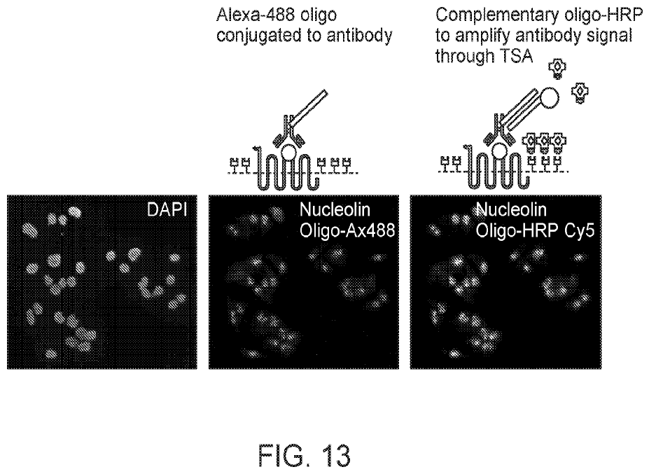

FIG. 13 shows how the present system can be used to detect nucleolin. In this experiment, results obtained from an Alexa Fluor® 488 conjugated anti-nucleolin antibody are compared to results produced using the present system (using a tyramide-Alexa Fluor® 488 conjugate and an oligonucleotide that is conjugated to HRP). As shown, the staining is significantly brighter using the present system.

### DOTA-Tyramide Synthesis and Loading with a Lanthanide

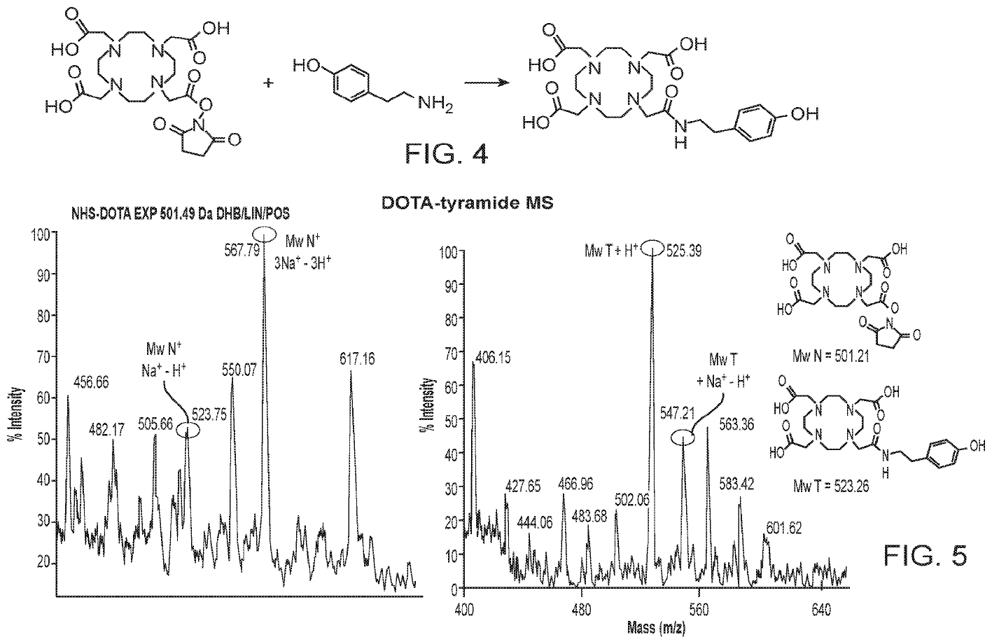

FIG. 4 illustrates how a tyramide-chelater conjugate can be produced. The reaction shown in FIG. 4 was done as follows: tyramine is not soluble in aqueous NaHCO₃ (0.2 M; pH 8.5), while the DOTA-NHS has JPF₆ and TFA, which is incompatible with the amine in the ligation reaction. In this reaction, 13.72 mg (100 umol) of tyramine was dissolved in 0.5 mL DMSO, and 76.15 mg (100 umol) DOTA-NHS was dissolved in DMSO first. The reagents were combined and then 0.5 mL of an aqueous solution of NaHCO₃ was mixed in to neutralize the acid. The resulting solution was incubated overnight at room temperature and then analyzed by MALDI-TOF(+). As shown in FIG. 5, the resulting solution of DOTA-tyramide is 66.7 mM (assuming 100% yield).

FIG. 6 illustrates how a tyramide-chelater conjugate can be loaded with a lanthanide, e.g., ¹⁵¹Eu or ¹⁶¹Dy. This reaction was done as follows: 3.5 μL of DOTA-tyramide (66.7 mM in water/DMSO at a 1:2 ratio) and 5 uL of a metal chloride (50 mM) were added to 91.5 uL of buffer (20 mM ammonium acetate, pH 6.0), at a molar ratio of 1:1.07. The solution was rotated at room temperature for 1 hr then frozen at −20° C. prior to analysis by MALDI-TOF(+). The results of the MALDI-TOF analysis are shown in FIG. 7.

### Cleavable Tyramide-Fluorophore Conjugates

As shown in FIG. 16, tyramide can be conjugated to a fluorescent dye via a disulfide bond. These disulfide bonds can be cleaved via the addition of TCEP, a strong reducing agent. This allows the removal of fluorescent dyes deposited via the HRP-Tyramide-dye reaction, after covalent deposition. Two examples of this reaction, with different dyes (Alexa Fluor® 488 and Cy3) are shown. Multiple cycles of HRP-Tyramide-dye deposition and removal allows for multiplexity in this system (>50-plex) over conventional methods (5-8-plex).

Representative examples of how certain aspects of the method can be implemented are shown in FIGS. 2-4.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method for analyzing a sample, comprising:
(a) obtaining:
i. a plurality of binding agents that are each linked to a different oligonucleotide; and
ii. a corresponding plurality of peroxidase-linked oligonucleotides, wherein each of the peroxidase-linked oligonucleotide specifically hybridizes with only one of the oligonucleotides of (a)(i);
(b) labeling the sample with the plurality of binding agents of (a)(i);
(c) specifically hybridizing a single peroxidase-linked oligonucleotide of the plurality of peroxidase-linked oligonucleotides of (a)(ii) with the sample, thereby producing complexes that comprise the peroxidase;
(d) treating the sample with at least one tyramide-label conjugate, wherein the peroxidase in the complexes produced in (c) activate tyramide in the conjugate and cause covalent binding of the label to the sample near the complexes;
(e) inactivating the peroxidase; and
(f) reading the sample to obtain data on the binding of the label.

2. The method of claim 1, wherein in step (c) the tyramide-label conjugate is a tyramide-mass tag conjugate and wherein the reading step (f) is done by a mass spectrometry-based method capable of detecting mass tags.

3. The method of claim 2, further comprising:
between steps (e) and (f) and without removing or inactivating the label that is associated with the sample in step (d), repeating steps (c), (d) and (e) multiple times, each repeat using a different peroxidase-linked oligonucleotide and a different tyramide-mass tag conjugate.

4. The method of claim 2, wherein the reading is done by multiplexed ion beam imaging (MIBI).

5. The method of claim 2, wherein the reading is done by mass cytometry.

6. The method of claim 3, wherein the method comprises repeating steps (c), (d) and (e) 5 to 100 times.

7. The method of claim 1, wherein the tyramide-label conjugate of step (c) is a tyramide-fluorophore conjugate that comprises a cleavable linker and wherein the reading of step (f) is done by fluorescence microscopy to produce an image showing the pattern of binding of the label to the sample.

8. The method of claim 7, wherein the method further comprises, after step (f):
(g) chemically removing the label that is associated with the sample in step (d) by cleaving a cleavable linker in the tyramide-fluorophore conjugate, thereby leaving the plurality of binding agents of (b) and their associated oligonucleotides still bound to the sample; and
(h) repeating steps (c), (d), (e) and (f) multiple times, each repeat using a different peroxidase-linked oligonucleotide and each repeat followed by step (g) except for the final repeat, to produce a plurality of images of the sample, each image corresponding to a peroxidase-linked oligonucleotide used in (c).

9. The method of claim 8, wherein step (h) comprises repeating steps (c), (d), (e) and (f) 5 to 100 times.

10. The method of claim 8, wherein:
the cleavable linker is cleavable by a reducing agent; and
in step (g) the label is removed using a reducing agent.

11. The method of claim 10, wherein the cleavable linker is a disulphide bond.

12. The method of claim 10, wherein the reducing agent is TCEP (tris(2-carboxyethyl)phosphine).

13. The method of claim 1, wherein the tyramide-label conjugate comprises a heavy metal and the reading step (f) is done by electron microscopy.

14. The method of claim 1, wherein the sample is treated with a single tyramide-label conjugate in step (d), thereby labeling the sample with a single label in step (d).

15. The method of claim 1, wherein the sample is treated with multiple tyramide-label conjugates in step (d), thereby labeling the sample with a combination of labels in step (d).

16.-20. (canceled)

21. A labeling system comprising:
(a) a plurality of binding agents that are each linked to a different oligonucleotide;
(b) a corresponding plurality of peroxidase-linked oligonucleotides, wherein each of the peroxidase-linked oligonucleotides specifically hybridizes with only one of the oligonucleotides of (a);
(c) a tyramide-label conjugate, wherein the tyramide of the conjugate is activatable by peroxidase treatment.

22. The labeling system of claim 21, wherein the tyramide-label conjugate comprises a mass tag, a heavy metal or a fluorophore.

23. The labeling system of claim 21, wherein the tyramide and label of the tyramide-label conjugate are joined by a cleavable linker.

24. A reagent system comprising:
(a) tyramide linked to a metal chelator or a heavy metal.

25. The system of claim 24, further comprising:
(b) a peroxidase-linked binding agent;

26-33. (canceled)
